(19) United States
(12) Patent Application Publication
(10) Pub. No.: US 2018/0333065 A1
(43) Pub. Date: Nov. 22, 2018

(54) SYSTEM AND METHOD FOR DETECTING TRAUMATIC BRAIN INJURY BY MEASUREMENTS OF AUTONOMIC NERVOUS SYTEM ACTIVITY

(71) Applicant: LIPIDRISK, LLC, The Woodlands, TX (US)

(72) Inventor: Jan M. TROUP, The Woodlands, TX (US)

(21) Appl. No.: 15/981,816

(22) Filed: May 16, 2018

Related U.S. Application Data
(60) Provisional application No. 62/507,517, filed on May 17, 2017.

(51) Int. Cl. A61B 5/0476; A61B 5/00

(52) U.S. Cl. CPC A61B 5/0476; A61B 5/4035; A61B 5/4064

(57) ABSTRACT

A system and method for assessing a patient includes a portable device for measuring a plurality of autonomic nervous system activities so as to form a baseline profile in a processor connected to the device. The baseline profile corresponds to an initial patient status before an impact event. A biomarker profile is determined by a parasympathetic ANS measurement and parasympathetic and sympathetic ANS measurement based on the baseline profile and a projected patient status for a traumatic brain injury. The device measures the plurality of autonomic nervous system activities after an impact event so as to form a test profile. The test profile is compared to the biomarker profile in a memory of the processor so as to form an injury index. When the injury index is equal or greater than a predetermined level, a traumatic brain injury is detected. An interactive display can indicate the traumatic brain injury.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention relates to detection of traumatic brain injury (TBI) in mammals. More particularly, the present invention relates to detection of traumatic brain injury in humans by measurements of the autonomic nervous system (ANS). The present invention also relates to the system and method for establishing ANS markers of TBI and detecting those ANS markers so as to set a proper medical treatment protocol.

### 2. Description of Related Art Including Information Disclosed Under 37 CFR 1.97 and 37 CFR 1.98

Traumatic brain injury or concussion is the leading cause of death in people under the age of 45 in the United States. Data from The Center for Disease Control (CDC) indicate that approximately every 22 seconds someone in the United States sustains a serious traumatic brain injury. Traumatic brain injuries can range from mild to moderate to severe. Traumatic brain injury (TBI) and concussion are used interchangeably. There are about 3.8 million sports and recreational related concussions in the United States every year with 1,365,000 emergency room visits and 52,000 deaths.

The conventional detection of a TBI is direct measurement of brain activity with electroencephalography (EEG). Electrodes on the scalp monitor voltage fluctuations due to neurons in the brain. The known electrophysiological monitoring can show reduced brain activity to confirm a TBI after an impact to the brain. Direct measurement of brain activity is not readily accessible, when patients are not located in a clinical setting. The access to EEG equipment is limited to hospitals and medical facilities with the proper support and personnel to work the equipment. Additionally, the equipment is expensive and not portable for locations away from hospitals and medical facilities.

In sports and athletics, a concussion protocol for diagnosis usually includes a series of verbal questions asked to an athlete, after an impact to the head. There is no EEG for direct brain activity measurement, especially in youth sports. The self-reported answers are used to determine whether a mild concussion or mild TBI or a serious TBI has occurred. Sometimes, the damage to the brain has immediately started, but the athlete may not immediately perceive the damage. Several days may pass with an ongoing neurometabolic cascade.

Several prior art patent applications address detecting a TBI of athletes without an EEG. U.S. Patent Publication No. 2015173666, published for Smith et al on 25 Jun. 2015, and U.S. Patent Publication No. 20160066847, published for Sales et al on 10 Mar. 2016, disclose wearable devices with sensors. A wearable helmet is fitted with various sensors to detect movement and posture.

U.S. Patent Publication No. 20130053652, published for Cooner on 28 Feb. 2013, addresses detecting TBI with wearable accelerometers on a helmet, during the physical activity. U.S. Pat. No. 9,326,737, issued to Simon on 3 May 2016, also relies on detecting body movement to determine the extent of injuries. PCT Patent Publication No. WO 2016094375, published for Torres on 16 Jun. 2016, describes a sensor system to detect changes in movement for detecting the extent of TBI or neurological disorders.

These prior art devices only monitor possible physical impacts to the brain or overall body movement and position without any measurement of actual brain activity. These devices still rely on diagnosing the TBI by verbal questions to the athlete. The TBI is only detected by self-reporting by the athlete, after any particular size impact was detected by the sensors or a particular deviation in the body position was detected by the sensors. The prior art devices only determine when the questions about TBI should be asked to the athlete. These systems still lack the reliability and consistency of an actual brain-related measurement.

The trauma of cerebral concussion or TBI generates compressive, tensile, and rotational forces resulting in diffuse axonal injury. Immediately following the injury, there is a sudden intracellular efflux of potassium and an influx of calcium ions producing a hypercalcemia condition in the brain. The concussed brain goes into a period of depressed metabolism with continued increases in calcium potentially impairing mitochondrial oxidative metabolism. The calcium accumulation can lead to cell death and disrupt neurofilaments and microtubules. The calcium accumulation is seen within hours of a concussion and persists for two to four days after an event. Additionally, cerebral swelling, as a result of calcium and sodium influx, occurs post concussion and further exposes the patient to additional risk. All of these physical and chemical factors change the neurometabolic status of the brain and have a pronounced effect on the autonomic nervous system (ANS). See Giza, C. C. and Hovda, D. A. The Neurometabolic Cascade of Concussion. J. Athletic Training. 2001, 36(3), 228-235.

Measuring aspects of the ANS (pulse wave changes, heart rate variability, and blood pressure) is known. Checking the heart rate of a patient or athlete is not a new process. U.S. Pat. No. 8,478,539, issued to Sieracki on 2 Jul. 2013, describes a generic sensor for ANS activity with processing to detect a predetermined physiological response. U.S. Patent Publication No. 20110245633, published for Goldberg et al. on 6 Oct. 2011, discloses a wearable sensor for oxygen saturation, heart rate, blood pressure, and galvanic skin response for detecting medical conditions. U.S. Patent Publication No. 20120245439, published for Andre et al. on 27 Sep. 2012, discloses another continuously worn wrist or arm sensor for various parameters, including oxygen saturation, heart rate, blood pressure, and galvanic skin response. U.S. Patent Publication No. 201500306340, published for Giap et al. on 29 Oct. 2015, discloses an overall hospital-based sensor system for detecting parameters and processing data for detecting physiological changes.

Besides monitoring the ANS for changes indicating an altered physiological state, the ANS measurements have been used to detect underlying medical conditions. U.S. Pat. No. 8,280,503, issued to Linderman on 2 Oct. 2012, biometric measurements on a glove for galvanic skin response disclose motor deficiencies related to underlying medical conditions. PCT publication No. WO2014199221 for Maarek on 18 Dec. 2014, ANS measurements are used to detect peripheral distal neuropathy in diabetic patients, not TBI or concussion.

Although aspects of ANS have been routinely measured, such as taking blood pressure readings and heart rate, the relationships between TBI and the ANS are only recently being investigated. For example, arterial stiffness and stroke volume are shown to be increased in concussed athletes. See La Fountaine et al., “Autonomic Nervous System Responses to concussion: Arterial Pulse Contour Analysis”, Frontiers in Neurology 7:13, 2016. Heart rate variability was also shown to be affected by TBI. The sympathovagal balance is increased in concussed athletes. See Abaji, Joseph Patrick, et al. “Persisting Effects of Concussion on Heart Rate Variability during Physical Exertion”, Journal of Neurotrauma. April 2016, 33(9): 811-817. Furthermore, abnormal ANS function due to TBI can be identified across different ANS measurements. See Goodman et al, “Autonomic Nervous System Dysfunction in Concussion (P01.265)”, Neurology (2013).

It is an object of the present invention to provide a system and method to detect a traumatic brain injury.

It is another object of the present invention to provide a system and method to detect a traumatic brain injury by measuring autonomic nervous system activity.

It is still another object of the present invention to provide a system and method to detect biomarkers of a traumatic brain injury in the autonomic nervous system.

It is still another object of the present invention to provide a system and method to screen measurements of the autonomic nervous system to detect a traumatic brain injury.

It is still another object of the present invention to provide a system and method to compare measurements of the autonomic nervous system before and after a potential traumatic brain injury.

It is an object of the present invention to provide a system and method to detect a traumatic brain injury without an electroencephalographic measurement.

It is an object of the present invention to provide a system and method to determine severity of a traumatic brain injury.

It is another object of the present invention to provide a system and method to determine severity of a traumatic brain injury by measuring autonomic nervous system activity.

It is still another object of the present invention to provide a system and method to compare autonomic nervous system activity and biomarkers of traumatic brain injuries.

It is still another object of the present invention to provide a system and method to screen autonomic nervous system activity for changes related to severity of traumatic brain injuries.

It is an object of the present invention to provide a system and method to determine severity of a traumatic brain injury without an electroencephalographic measurement.

It is an object of the present invention to provide a portable system and method to detect a traumatic brain injury.

It is another object of the present invention to provide a system and method using a handheld device to detect a traumatic brain injury.

It is another object of the present invention to provide a system and method using a wireless network connected to a handheld device to detect a traumatic brain injury.

These and other objects and advantages of the present invention will become apparent from a reading of the attached specification.

### BRIEF SUMMARY OF THE INVENTION

Embodiments of the present invention include a method for assessing a patient. A baseline profile is formed by measuring a plurality of autonomic nervous system activities. The baseline profile corresponds to the patient before any impact event, such taking these measurements before an athlete participates in a practice or actual game. The baseline profile is used to form a biomarker profile corresponding to autonomic nervous system changes, if the patient was injured. Only particular changes to the autonomic nervous system can be used to form the biomarker profile. The method further includes measuring the same plurality of autonomic nervous system activities so as to form a test profile. The test profile corresponds to an injured patient status after any impact event or any suspected impact event. The test profile is compared to the biomarker profile so as to form an injury index based on matches between the test profile and the biomarker profile. The method detects a traumatic brain injury when the injury index is equal or greater than a predetermined level. Instead of relying on self-reports of dizziness or concussion, the method provides a reliable diagnosis of possible brain injury.

In some embodiments, the plurality of autonomic nervous system activities can include pulse rate variability, pulse wave changes, cardiac output, blood pressure changes, bioimpedance, and sweat loss. The biomarker profile sets the amount, number, and combination of changes to the autonomic nervous system to accurately characterize a traumatic brain injury to a particular patient. The present invention includes a biomarker profile comprised of a parasympathetic ANS measurement and parasympathetic and sympathetic ANS measurement. In particular, the biomarker profile is comprised of a higher SDNN and either lower LF or LF/HF measurements. In another embodiment, the biomarker profile is comprised of higher SDNN, lower LF, and lower LF/HF measurements. After a suspected impact event, a patient can be assessed so that the patient can be withdrawn from the playing field or referred for further medical attention. The method can be completed immediately after a suspected impact event or repeated at set time intervals after a suspected impact event.

The method can also include determining severity of the traumatic brain injury. A severity profile, based on the baseline profile, corresponds to a plurality of levels of seriousness of a traumatic brain injury. The injury index can be compared to the severity profile and matched to at least one level of seriousness. In some embodiments, severity of the traumatic brain injury requires measurement of multiple autonomic nervous system activities. In particular, a Valsalva test and a K30/15 standing test should be part of the base profile and any corresponding severity profile.

Embodiments of the present invention also include the system for assessing a patient. The system is portable so that a hospital or other medical facility is not required to perform the method of the present invention. The system can include a processor, a memory connected to the processor, and a portable measuring device for a plurality of autonomic nervous system activities. The portable measuring device connects to the processor so that data from the measuring device is communicated to the processor to form the baseline profile, the biomarker profile, the test profile, and the injury index, of the method. These profiles and the predetermined level can be stored in the memory. In some embodiments, the portable measuring device can be a finger pulse oximeter connected by a cable to a smartphone or tablet with the processor. Alternatively, the portable measuring device can be connected to the processor by a wireless network (Bluetooth, wifi, etc.). The processor may be the server connected through the wireless network through a mobile device, like a smartphone or tablet computer. In some versions, there is an interactive display connected to the processor, like the screen of a smartphone. The screen can show the profiles and the result of whether a traumatic brain injury was detected.

### BRIEF DESCRIPTION OF THE SEVERAL VIEWS OF THE DRAWINGS

FIG. 1 is a schematic view of an embodiment of the system and method of the present invention for assessing a patient.

FIG. 2 is a schematic view of an interactive display showing the profiles and indicators of the injury index, according to the method of the present invention.

### DETAILED DESCRIPTION OF THE INVENTION

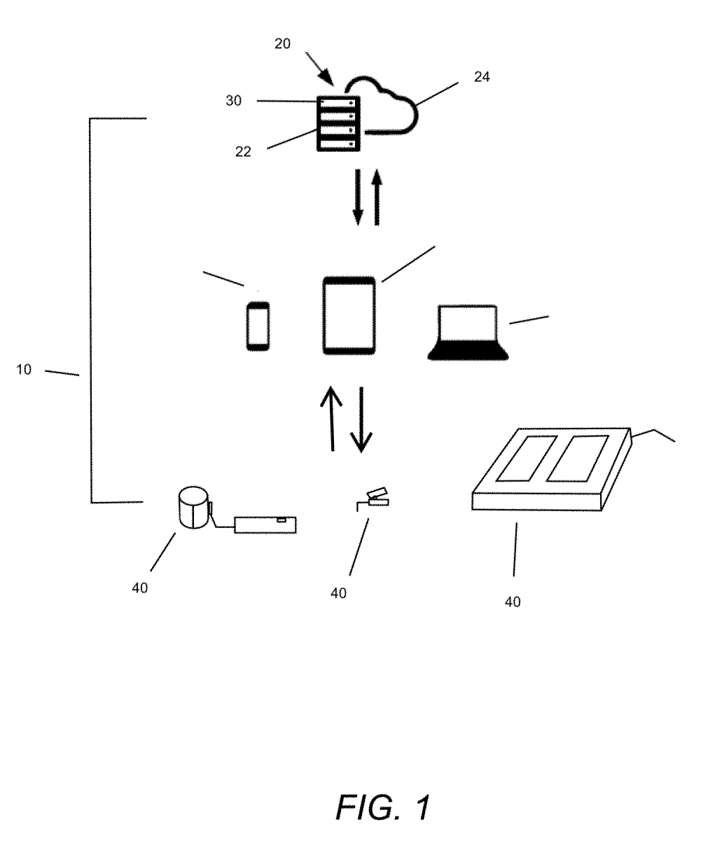

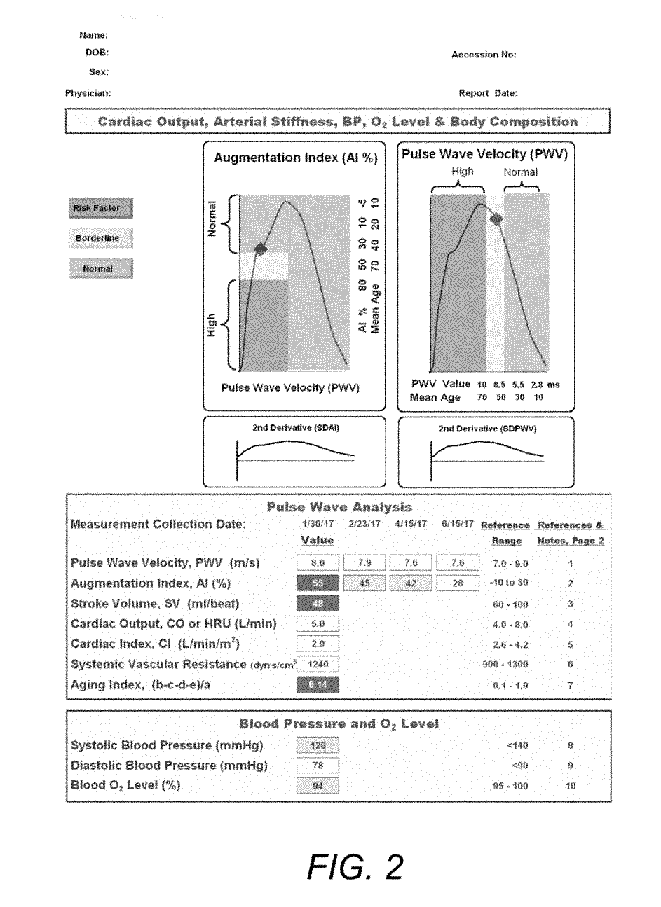

FIGS. 1-2 show embodiments of the system 10 and method for assessing a patient. The system 10 and method reliably determine whether the patient has suffered a traumatic brain injury (TBI) or concussion. The conventional diagnosis relied on self-reporting by the patient, such as asking the patient to describe symptoms of dizziness or disorientation. These subjective metrics cannot be standardized or quantified in a meaningful way to diagnose TBI or severity of TBI. In contrast, an electroencephalography (EEG) reading directly measures brain activity. The EEG can detect any changes in brain activity due to a physical impact to the brain. However, the equipment for an EEG is not readily accessible for many situations. The present invention is a system 10 and method that are more reliable than the self reporting and more practical than EEG equipment.

FIG. 1 shows the system 10 based on the autonomic nervous system (ANS). The other parts of the nervous system, besides the brain, can be assessed. The system 10 includes a processor 20, a memory 30 connected to the processor 20, and a portable measuring device 40 for a plurality of autonomic nervous system activities. FIG. 1 shows an embodiment of the processor 20 as a server 22 with the memory 30 as part of the server 22. A wireless network 24 connects the processor 20 to at least one portable measuring device 40. FIG. 1 also shows an embodiment with the portable measuring device 40 connected to a mobile device 50, and the processor 20 connects through the mobile device 50. The mobile devices 50 can be smartphones, tablet computers or laptop computers. These mobile devices 50 can use Bluetooth, direct wired connection or other wireless networks to connect to the portable measuring devices 40. The portable measuring devices 40 can be a blood pressure cuff, a finger pulse oximeter, or a contact pad for bioimpedance. In some embodiments, the processor 20 can be incorporated in the mobile device 50, if there is sufficient memory in the mobile device 50. A laptop as a mobile device may have the capacity to have a memory 30, but a smartphone may require wireless connection to a server 22. The portable measuring devices 40 may also be smart devices on their own with connectivity direct to the server 22 through the wireless network 24.

The system 10 is portable, even when a mobile device 50 is incorporated into the system 10, and the system 10 can be powered by batteries. A power source can be mobile and portable without requiring a wall socket in a medical facility. The system 10 is more versatile to be compatible with ANS measurements for brain activity without the EEG equipment. In the embodiments with the mobile devices 50, there can be an interactive display connected to the processor 20 through the mobile device 50 so that a baseline profile, a biomarker profile, a test profile, and an injury index can be visible to a doctor or patient. When a TBI is detected, the interactive display can show the diagnosis and provide a warning. If a patient is a player in an athletic contest, the patient can be removed from the field of play when the interactive display indicates that a TBI was detected.

The memory 30 can also be associated with the mobile device 50, when the processor 20 is also associated with the mobile device 50. When there is no readily available access to a wireless network, it may be necessary to have a mobile device 50 with the capacity to act as the processor 20, such as a corresponding memory in the mobile device 50. The predetermined levels of the injury index can also be stored in the memory 30 of either location in the mobile device 60 or the processor 20.

Embodiments of the present invention include the method for assessing a patient with the system 10 of FIG. 1. The method includes measuring a plurality of autonomic nervous system activities so as to form a baseline profile, determining a biomarker profile based on the baseline profile, measuring the plurality of autonomic nervous system activities so as to form a test profile, comparing the test profile to the biomarker profile so as to form an injury index based on matches between the test profile and the biomarker profile, and detecting a traumatic brain injury when the injury index is equal or greater than a predetermined level.

The baseline profile corresponds to an initial patient status before an impact event. The baseline profile is created by measurements of ANS before a practice or competition for each athlete. The biomarker profile corresponds to a projected patient status for a traumatic brain injury. The biomarker profile is based on autonomic nervous system changes, if the patient was injured. Only particular changes to the autonomic nervous system can be used to form the biomarker profile of the present invention. The biomarker profile sets the amount, number, and combination of changes to the autonomic nervous system to accurately characterize a traumatic brain injury for a particular patient. Although heart rate is a measurement for other ANS measurements and although heart rate is already known to be measured after a potential injury, there is no biomarker profile generated from heart rate measurements to screen for TBI or a concussion as in the present invention. A TBI or concussion cannot be diagnosed by simply taking the heart rate of a patient.

Test data supports the efficacy of a biomarker profile of the present invention. Prior art indicates that there are changes in the ANS related to TBI, such as arterial stiffness and stroke volume, heart rate variability, and sympathovagal balance, but there is a wide range of ANS measurements. The ANS covers both sympathetic (fight or flight) responses and parasympathetic (regulation of the body's unconscious actions) responses, so there is only a general disclosure of ANS being affected by TBI and concussions. Because changes in ANS may result from other stimuli besides TBI, there is no knowledge for distinguishing these other changes in ANS and the changes in ANS indicating TBI. The present invention now identifies the biomarker profile of ANS measurements for reliably detecting TBI.

In a preliminary study, pulse oximeter data for pulse wave (PW) analysis and other autonomic nervous system (ANS) measurements were used in a small study to compare six healthy patients ages 14 to 71 with two patients ages 14 and 15 that had confirmed concussions. Due to the differences in age between the healthy control group and the younger concussed patients and the high correlation between age and pulse wave analysis, not all results were comparable between “No TBI” and “Confirmed TBI” in this small sample group. Although preliminary, the results show trends that appear to be significant. Further study in a controlled athletic environment can further support the present disclosures, with more control for age and the TBI events.

TABLE 1

| | | | 6 Patients, | 2 Patients | |
|---|---|---|---|---|---|
| | | | Ages 14-71 | Ages 14 & 15 | |
| | | | No TBI | Confirmed TBI | |
| | Reference | | Average | Average | |
| ANS Measurements | Value | | Values | Values | p = |
| SDNN, Parasym- | >40 | ms | 48 | 74 | 0.02 * |
| pathetic | | | | | |
| RMSSD, Parasym- | 35-65 | ms | 55 | 73 | 0.07 |
| pathetic | | | | | |

| | | | | |
|---|---|---|---|---|
| Low Frequency | 22-46% | 49 | 26 | 0.03 * |
| (LF) - Sympathetic | | | | |
| High Frequency | 22-34% | 30 | 37 | 0.08 |
| (HF) - Parasym- | | | | |
| pathetic | | | | |
| LF/HF Sym/Para | <2.0 | 1.7 | 0.7 | 0.04 * |
| balance | | | | |

* Significant Result with p < 0.05

In Table 1, the present invention identifies particular biomarkers for the biomarker profile. The standard deviation of normal to normal intervals (SDNN) is an ANS measurement of pulse rate variability. SDNN is a measure of parasympathetic activity. The low frequency fraction (LF) is another ANS measurement of pulse rate variability. LF is a measure of both parasympathetic and sympathetic activity. The ratio of low frequency to high frequency (LF/HF) is another ANS measurement of pulse rate variability. LF/HF also measures parasympathetic and sympathetic activity as a balance of sympathetic to parasympathetic balance.

The ANS impairment in sympathetic sudomotor function has been observed, and Table 1 confirms this sympathetic impairment in the lower LF sympathetic measurement and the LF/HF balance results. However, the SDNN results shows that the parasympathetic activity increased. These distinctions between different ANS measurement are the first preliminary results showing an impairment in the sympathetic autonomic nervous system and a positive parasympathetic reaction to a TBI or concussion.

Table 1 shows that among several ANS measurements, SDNN, LF, and LF/HF, were determined to be significant with p<0.05. The present invention includes a biomarker profile comprised of a parasympathetic ANS measurement and parasympathetic and sympathetic ANS measurement. In particular, the biomarker profile is comprised of a higher SDNN and either lower LF or LF/HF measurements. In another embodiment, the biomarker profile is comprised of higher SDNN, lower LF, and lower LF/HF measurements.

Embodiments of the present invention can also include a biomarker profile comprised of other ANS measurements. Table 1 also shows increases at a lower statistical significance level, including high frequency (HF) pulse rate variability, a marker of parasympathetic activity and the root mean squared difference between adjacent N-N intervals (RMSSD), as a measure of parasympathetic activity. These ANS measurements of parasympathetic activity (RMSSD and HF) are less significant than the SDNN. The unconscious positive ANS reaction to a concussion cannot be measured by all parasympathetic activity ANS measurements yet. Further investigation may reveal how to incorporate HF and RMSSD into a biomarker profile. If more robust differences can be isolated, then these other ANS measurements on the parasympathetic activity side may also be used to contribute to a more precise biomarker profile.

TABLE 2
Following Assays Are Not Significant
Due to the Lower Age of TBI Patients

| | | | 6 Patients, | 2 Patients |
|---|---|---|---|---|
| | | | Ages 14-71 | Ages 14 & 15 |
| Pulse Wave Analysis | | | No TBI | Confirmed TBI |
| SI, Pulse Wave Velocity | <11 | m/sec | 8.5 | 5.9 |
| Aix, Augmentation Index | <1.4 | | 1.3 | 1.0 |
| PEP, Systolic Pre- | <110 | ms | 124 | 124 |
| ejection Peroid | | | | |
| LVET, Left Ventricle | >300 | ms | 338 | 436 |
| Ejection Time | | | | |
| PEP/LVET | <0.4 | | 0.4 | 0.3 |
| b/a, Left Ventricle | −0.82 to −0.46 | | −0.9 | −1.2 |
| Ejection Power | | | | |
| Systolic Time | 200-500 | ms | 276 | 339 |

| | | | |
|---|---|---|---|
| spO2% | >95% | 97 | 99 |

Table 2 shows the pulse wave analysis for “No TBI” and “Confirmed TBI”. There are large differences in the pulse wave velocity (SI) and augmentation index (Aix) between the concussed patients and the control group but due to the large average age differences in these groups the results are discounted. It is expected that as a person ages that the SI and Aix values will increase due to stiffening of the arteries with age. This stiffening, due to atherosclerosis and loss of tissue flexibility, makes the reflected wave of the pulse wave return faster and increases the velocity of the pulse. Additionally, the augmentation (Aix) of the systolic wave generated by the reflected wave also increases with age. Although previously known to be significant in determining TBI or concussion in controlled pre and post-concussion testing in the prior art, the current preliminary study may be too small to confirm S1 and Aix as ANS measurements in the biomarker profile of the present invention.

Other ANS measurements in Table 2 include cardiac function as measured by systolic pre-ejection period (PEP), left ventricle ejection time (LVET), the PEP/LVET ratio, the left ventricle ejection power (b/a) and the systolic time. These measures of cardiac performance are also highly age dependent. So again, the current preliminary study may be too small to confirm these measurements in the biomarker profile of the present invention. Further investigation may be able to determine additional embodiments of the present invention for increasing precision based on pulse wave analysis of ANS measurements.

The test profile corresponds to an injured patient status after the impact event. Subsequent measurements of ANS, after the baseline profile is created, form the test profile. Various embodiments include the step of measuring the plurality of autonomic nervous system activities so as to form the test profile is within thirty minutes after an impact event, days after the impact event, immediately after the impact event, and at repeated time intervals for a series of test profiles. Each test profile can be used to track when the TBI was detectable after the impact event. FIG. 2 shows an interactive display with a baseline profile on Jan. 17, 2030 and subsequent test profiles at Feb. 23, 2017, Apr. 15, 2017, and Jun. 15, 2017. The reference range for normal is also shown. There are markings to indicate when a particular measurement of ANS is shows a change, including changes corresponding to a match with the biomarker profile.

Embodiments of the present invention include the plurality of autonomic nervous system activities as pulse rate variability, pulse wave changes, cardiac output, blood pressure changes, bioimpedance, and sweat loss. Measurements of these activities are the measurements of ANS. The baseline profile, biomarker profile, test profile, injury index, and predetermined level all correspond to the selection of the particular measurements of ANS. The present invention can choose the amount, number, and combination of measurements of these activities to set the profiles, and consequently the injury index and predetermined level. The current embodiment includes the biomarker profile being comprised of a parasympathetic ANS measurement of pulse rate variability and blended parasympathetic and sympathetic ANS measurement of pulse rate variability. In particular, the biomarker profile can include SDNN and either LF or LF/HF measurements. In another embodiment, the biomarker profile is comprised of SDNN, LF, and LF/HF measurements.

FIG. 2 shows an alternative embodiment the biomarker profile to include pulse wave analysis, in addition to the biomarker profile being comprised of a parasympathetic ANS measurement and blended parasympathetic and sympathetic ANS measurement. As disclosed in Table 2, there are pulse wave changes related to TBI, but those changes are not yet robust. Other embodiments may still currently include ANS measurements of pulse wave changes related to arterial stiffness. Pulse Wave Analysis includes the pulse wave velocity (SI or PWV) and augmentation index (Aix or AI), stroke volume (SV), systolic pre-ejection period (PEP), left ventricle ejection time (LVET), the PEP/LVET ratio, the left ventricle ejection power (b/a) and the systolic time. Eventually, some of these pulse wave analysis ANS measurements may be incorporated into the biomarker profile.

Still embodiments include cardiac output as ANS measurements incorporated into the biomarker profile. FIG. 2 shows the measuring Cardiac output (CO) in L/min, Cardiac index (CI) in L/min/m2, Systematic vascular resistance (SVR) in dyn·s/cm, and Aging index in (b−c−d−e)a. The activity can also be blood pressure changes, as measured by Systolic blood pressure in mm Hg, Diastolic blood pressure in mm Hg. Other possible ANS measurements include bioimpedance, wherein the steps of measuring include measuring Body total water in percent, and sweat loss, wherein the steps of measuring include performing a quantitative sudomotor axon reflex test (QSART) and measuring postganglionic sudomotor system changes.

In the present invention, the baseline profile is comprised of ANS measurements of pulse rate variability. There are changes in the pulse rate variability, but the present invention accomplishes more than merely recording the changes. It is already known to measure changes in activity. The present invention is a method that advances to screen or filter the changes so that only changes relevant to TBI are identified. The prior art of identifying general ANS differences changes after impact cannot determine whether those differences are related to brain damage of a concussion or TBI.

The present invention measures a particular combination for the most relevant and most accurate baseline profile, biomarker profile, and test profile. The injury index and predetermined levels demonstrate reliability in indicated a traumatic brain injury or concussion.

Various other factors can also contribute to the baseline profile, including Body mass index (BMI) in cm/kg, Body fat in percent, Blood oxygen level in percent saturation, Height to weight in ratio, and Basal metabolic rate in calories. Although not necessarily ANS measurements, these factors contribute to the efficacy of the injury index and predetermined level for a particular patient.

Still another embodiment of the method of the present invention is detecting severity of the TBI. In addition to confirming TBI, the method of the present invention can be used to determine the seriousness of the TBI. The method further includes determining a severity profile corresponding to a plurality of levels of seriousness of the traumatic brain injury. Similar to the biomarker profile, the severity profile sets the levels of seriousness. Once past a threshold, the amount of change of activity can be used to further characterize the TBI for guiding treatment decisions or patient observation. Based on the baseline profile, the severity profile is compared to the injury index. The injury index matches at least one level of seriousness of the traumatic brain injury. Thus, the TBI can be confirmed and characterized for more effective treatment protocols.

In the current embodiments of detecting severity, one of the activities for the steps of measuring is performing a Valsalva test so as to determine a Valsalva result or performing a K30/15 standing step test so as to determine a K30/15 standing step result. These two particular activities are particularly relevant to detecting severity, as such the baseline profile of this embodiment should include at least one of the Valsalva result and the K30/15 standing step result.

The present invention provides a system and method to detect a traumatic brain injury without an EEG and without relying on self-reported information from the patient. The present invention is more cost efficient and accessible than direct measurement of brain activity, and the detection is no longer reliant on verbal responses from the patient. Especially for serious injuries when the patient cannot speak, it is important to quick assess the patient.

The system and method measure autonomic nervous system (ANS) activity, including before and after impacts suspected to cause a TBI or concussion. Although changes in ANS activity have already been observed after TBI, there has been no method to use those changes for any diagnosis. For example, taking a heart rate measurement and noticing a change in heart rate after an impact could happen for a variety of different physiological and psychological reasons. Similarly, taking a Valsalva test and noticing a change in a Valsalva result after an impact could also happen for a different variety of different physiological and psychological reasons. The present invention detects biomarkers of a traumatic brain injury in the autonomic nervous system. The changes in ANS activity are screened so that not just any changes to any ANS activity measurements detect a TBI. Prior art systems already measure some ANS activity, but these measurements are not used to form a biomarker profile, and test profiles are not created for comparison to the biomarker profile. The prior art literature identifying changes to ANS activity do not identify the amount, number, and combination of changes to reliably detect a TBI. The present invention provides the system and method to compare measurements of the autonomic nervous system before and after a potential traumatic brain injury.

Embodiments of the present invention can determine severity of a traumatic brain injury (TBI), in addition to confirming a TBI. Similar to the biomarker profile, there is no severity profile to define the levels of seriousness of a TBI according to measurements of ANS activities. The present invention further screens for the level of seriousness with the injury index of the test profile after an impact. The severity of a TBI can now be determined without an electroencephalographic measurement.

The system is portable so as to be available at athletic events on all levels, from youth to professional sports. A handheld device, such as a smartphone or tablet can be retrofit for compatibility with a device for measuring ANS activity. The smartphone with wireless connection to a service can become a system to detect a traumatic brain injury. A finger pulse oximeter plugged into a tablet can become a potentially life-saving diagnostic tool. The present invention enables known components to perform an innovative and beneficial method for detecting a TBI and determining severity of a TBI.

The foregoing disclosure and description of the invention is illustrative and explanatory thereof. Various changes in the details of the described method can be made without departing from the true spirit of the invention.

I claim:

1. A method for assessing a patient, the method comprising the steps of:
measuring a plurality of autonomic nervous system (ANS) activities so as to form a baseline profile, said baseline profile corresponding to an initial patient status before an impact event;
determining a biomarker profile based on said baseline profile, said biomarker profile corresponding to a projected patient status for a traumatic brain injury;
measuring said plurality of autonomic nervous system activities so as to form a test profile, said test profile corresponding to an injured patient status after said impact event;
comparing said test profile to said biomarker profile so as to form an injury index based on matches between said test profile and said biomarker profile; and
detecting a traumatic brain injury when said injury index corresponds to a predetermined level.

2. The method for assessing a patient, according to claim 1,
wherein said plurality of autonomic nervous system activities is comprised of at least one of a group consisting of pulse rate variability, pulse wave changes, cardiac output, blood pressure changes, bioimpedance, and sweat loss.

3. The method for assessing a patient, according to claim 1,
wherein said biomarker profile is comprised of a parasympathetic ANS measurement and a blended parasympathetic and sympathetic ANS measurement.

4. The method for assessing a patient, according to claim 3,
wherein said parasympathetic ANS measurement is the standard deviation of normal to normal intervals (SDNN).

5. The method for assessing a patient, according to claim 4,
wherein said blended parasympathetic ANS measurement is low frequency fraction (LF).

6. The method for assessing a patient, according to claim 4,
wherein said blended parasympathetic ANS measurement is ratio of low frequency to high frequency fraction (LF/HF).

7. The method for assessing a patient, according to claim 1,
wherein said biomarker profile is comprised of a parasympathetic ANS measurement, a first blended parasympathetic and sympathetic ANS measurement, and a second blended parasympathetic and sympathetic ANS measurement.

8. The method for assessing a patient, according to claim 7,
wherein said parasympathetic ANS measurement is the standard deviation of normal to normal intervals (SDNN),
wherein said first blended parasympathetic ANS measurement is low frequency fraction (LF), and
wherein said second blended parasympathetic ANS measurement is ratio of low frequency to high frequency fraction (LF/HF).

9. The method for assessing a patient, according to claim 4,
wherein the step of detecting comprises said SDNN of said injury index being greater than said predetermined level.

10. The method for assessing a patient, according to claim 9,
wherein said blended parasympathetic ANS measurement is low frequency fraction (LF), and
wherein the step of detecting comprises said LF of said injury index being less than said predetermined level.

11. The method for assessing a patient, according to claim 9,
wherein said blended parasympathetic ANS measurement is ratio of low frequency to high frequency fraction (LF/HF), and
wherein the step of detecting comprises said LF/HF of said injury index being less than said predetermined level.

12. The method for assessing a patient, according to claim 3,
wherein said biomarker profile is further comprised of at least one of a group consisting of high frequency (HF) pulse rate variability and the root mean squared difference between adjacent N-N intervals (RMSSD).

13. The method for assessing a patient, according to claim 3,
wherein said biomarker profile is further comprised of at least one of a group consisting of pulse wave changes, cardiac output, blood pressure changes, bioimpedance, and sweat loss.

14. The method for assessing a patient, according to claim 13,
wherein said pulse wave changes is comprised of at least one of a group consisting of pulse wave velocity (SI or PWV), augmentation index (Aix or AI), stroke volume (SV), systolic pre-ejection period (PEP), left ventricle ejection time (LVET), the PEP/LVET ratio, the left ventricle ejection power (b/a) and the systolic time.

15. The method for assessing a patient, according to claim 1, further comprising the steps of:
determining a severity profile based on said biomarker profile, said severity profile corresponding to a plurality of levels of seriousness of said traumatic brain injury;
comparing said injury index to said severity profile; and
matching said injury index to at least one level of seriousness of said traumatic brain injury of said severity profile.

16. The method for assessing a patient, according to claim 15,
wherein the step of measuring said plurality of autonomic nervous system activities is comprised of: performing a Valsalva test so as to determine a Valsalva result, said baseline profile being comprised of said Valsalva result,
wherein said biomarker profile is comprised of said Valsalva result,
wherein said severity profile is comprised of said Valsalva result, and
wherein the step of measuring said plurality of autonomic nervous system activities is comprised of: performing a K30/15 standing step test so as to determine a K30/15 standing step result, said Valsalva result being being comprised of said K30/15 standing step result.

17. A system for assessing a patient, comprising:
a processor;
a memory being comprised of a computer program code for one or more programs; and
a portable measuring device for a plurality of autonomic nervous system activities, said portable measuring device being connected to said processor,
wherein said processor forms said baseline profile, said biomarker profile, said test profile, and said injury index, according to claim 1, and
wherein said predetermined level is stored in said memory,

18. The system for assessing a patient, according to claim 17,
wherein said portable measuring device is connected to said processor by a wireless network.

19. The system for assessing a patient, according to claim 17,
wherein said portable measuring device is powered by a battery.

20. The system for assessing a patient, according to claim 17, further comprising:
an interactive display connected to said processor, said baseline profile, said biomarker profile, said test profile, and said injury index being visible on said interactive display,
wherein said processor detects a traumatic brain injury when said injury index corresponds to a predetermined level, said interactive display indicating whether said traumatic brain injury is detected.
